(19) United States
(12) Patent Application Publication
(10) Pub. No.: US 2011/0053143 A1
(43) Pub. Date: Mar. 3, 2011

(54) METHODS AND KITS FOR DETERMINING VIRAL LOAD IN CLINICAL SAMPLES

(75) Inventors: Brian Lowe, Olney, MD (US); Dominic O'Neil, Alexandria, VA (US); Irina Nazarenko, Gaithersburg, MD (US); Dirk Loeffert, Duesseldorf (DE)

(73) Assignee: QIAGEN GAITHERSBURG INC., Gaithersburg, MD (US)

(21) Appl. No.: 12/875,533

(22) Filed: Sep. 3, 2010

Related U.S. Application Data
(60) Provisional application No. 61/239,530, filed on Sep. 3, 2009.

(51) Int. Cl. C12Q 1/70

(52) U.S. Cl. 435/5

(57) ABSTRACT

Methods and kits for determining load of an infectious agent in a sample are described, comprising performing at least one hybridization assay and calculating the load of the infectious agent in the sample from a detected nucleic acid. In particular, the methods and kits disclosed determine the load of human papillomavirus (HPV) in a sample.

### CROSS REFERENCE TO RELATED APPLICATIONS

This application claims priority from U.S. Provisional Patent Application Ser. No. 61/239,530 (filed Sep. 3, 2009), the contents of which are incorporated herein by reference in their entireties. A PCT application entitled “Methods and Kits for Determining Viral Load in Clinical Samples” (filed concurrently herewith on Sep. 3, 2010) is also incorporated herein by reference in its entirety.

### BACKGROUND OF INVENTION

1. Field of the Invention

The disclosure relates to methods and kits for determining infectious agent load in a sample.

2. Description of Related Art

Numerous diseases and conditions are associated with the presence of infectious agents, for example: ulcers and gastritis (helicobacter pylori infection), acquired immune deficiency syndrome (human immunodeficiency virus), anal and genital warts (herpes simplex virus). Although the presence of such infectious agents is sometimes sufficient to diagnose the diseased state, it often is not.

One example is cervical cancer. Infection by high-risk, HPV is a necessary cause of cervical cancer. Cervical cancer screening assays, such as the FDA-approved, DIGENE HC2 TEST, detect a group of 13 high-risk HPV types. Other genotyping assays detect multiple HPV types, including HPV 16 and 18, the two types most prevalent in cervical cancer. These molecular assays for HPV DNA have excellent negative predictive value; however, there are many cases where the HPV infection does not result in cancer or severe lesions. Therefore, new assays and biomarkers with the potential for higher positive predictive value are being investigated.

One of the biomarkers proposed to predict progression of the disease is HPV viral load. Although high HPV load is often associated with precursor lesions, only HPV 16 load was shown to predict the incidence of disease. Other studies indicate that viral load assessment had no added value over cytology and that testing for high load levels may not be clinically useful.

While the value of viral load as a biomarker for cervical cancer is not entirely clear, the assessment of HPV load in clinical samples is important for the development of accurate, clinical assays. The HPV load may impact carry-over contamination during the liquid-handling processes of an assay, especially during sample processing. The HPV load may also impact the analytical specificity of detection probes. The specificity of the probes is usually a function of the concentration of the potential cross-reactive Hpv types. Although the HPV load has been widely studied, the absolute amount of HPV DNA in clinical specimens is rarely reported. Most studies are based on quantitative PCR and report HPV copies per cell, relative to a human reference gene. The range of HPV load in specimens is reported to be between less than one copy per cell to more than 10⁴ copies per cell. There are only few studies, however, where the total amount of HPV per specimen is provided or can be calculated and the reported HPV load in such studies ranges broadly, between 10² and 10¹⁴ copies per specimen.

Therefore, there is a need for methods and kits for accurately determining the load of infectious agents, in particular HPV.

### SUMMARY OF INVENTION

The present disclosure relates to materials and methods for determining the load of a infectious agent in a sample material and correlating the load of that infectious agent with development or progression of a disease state.

In one aspect, a method of determining the load of a infectious agent in a sample is provided, said method comprising: performing at least one hybridization assay with one or more infectious agent nucleic acid or fragment thereof in the sample; detecting resultant hybridized infectious agent nucleic acid or fragment thereof; and calculating the load of the infectious agent in the sample from detected nucleic acid or fragment thereof.

In another aspect, a method for determining incidence of a infectious agent-related disease in a subject is provided, the method comprising: performing at least one hybridization assay with one or more infectious agent nucleic acid or fragment thereof in the sample; detecting resultant hybridized infectious agent nucleic acid or fragment thereof; calculating the load of the infectious agent in the sample from detected nucleic acid or fragment thereof; and correlating the load of the infectious agent in the sample to the incidence of the infectious agent-related disease.

In another aspect, a method for determining progression of a infectious agent-related disease in a subject is provided, the method comprising: providing a sample of diseased tissue from the subject at a first endpoint and a second endpoint; performing at least one hybridization assay with one or more infectious agent nucleic acid or fragment thereof in each sample of diseased tissue; detecting resultant hybridized infectious agent nucleic acid or fragment thereof; calculating the load of the infectious agent in the sample from each endpoint from the detected nucleic acid or fragment thereof; comparing the load of the infectious agent correlating the load of the infectious agent in the sample to the incidence of the infectious agent-related disease.

In another aspect, the infectious agent is a virus.

In another aspect, the infectious agent is a high-risk human papillomavirus.

In another aspect, the infectious agent-related disease is cervical cancer.

In another aspect, a method for determining the viral load in a sample is provided, the method comprising: performing at least one hybridization assay with one or more human papillomavirus DNAs or fragments thereof in the sample; detecting resultant hybridized HPV DNA or fragments thereof; and calculating the viral load from detected hybridized HPV DNA or fragments thereof.

In another aspect, a method for determining incidence or progression of human papillomavirus-related disease in a subject is provided, the method comprising: performing at least one hybridization assay with one or more human papillomavirus DNAs or fragments thereof in the sample; detecting resultant hybridized human papillomavirus DNA or fragments thereof; calculating viral load from detected hybridized human papillomavirus DNA or fragments thereof; and correlating the viral load to the incidence or progression of the human papillomavirus-related disease.

### BRIEF DESCRIPTION OF THE FIGURES

FIG. 1A shows the plot of signal (RLU) per HPV plasmid input (copies).

FIG. 1B shows the plot of the linear range of the assay.

FIG. 2 shows distribution histograms of RLU results with bins of 100,000 RLU for two populations (China, n=569; California, n=501). Data labels give percent of specimens within each bin.

FIG. 3 shows the plot of signal (RLU) versus HPV copies per assay for dilutions of two clinical specimens and the HPV 16 plasmid (circle, specimen ID 360; triangle, specimen ID 495; diamond, plasmid).

FIG. 4 shows box-and-whisker plots that indicate the signal RLU for each cytological diagnosis. Media, maxima, minima and inter-quartile range are shown.

### DETAILED DESCRIPTION

The present disclosure relates to materials and methods for determining the load of an infectious agent in a sample material and correlating the load of that infectious agent with development or progression of a disease state.

In one aspect, a method of determining the load of a infectious agent in a sample is provided, said method comprising: performing at least one hybridization assay with one or more infectious agent nucleic acid or fragment thereof in the sample; detecting resultant hybridized infectious agent nucleic acid or fragment thereof; and calculating the load of the infectious agent in the sample from detected nucleic acid or fragment thereof. Said method can be applied to a number of uses, including but not limited to: determining the incidence or progression of a disease associated with the infectious agent; comparing populations for incidence of a disease associated with the infectious agent; triaging subjects for treatment or further monitoring; and triaging samples for further analysis.

Samples include, but are not limited to, clinical specimens or cultures, such as cell cultures, fluids (e.g. urine), solids (e.g., stool) or tissue samples; plant tissue or seed; as well as liquid and solid food and feed products and ingredients such as dairy items, vegetables, meat and meat byproducts, and waste. Such samples may be drawn from any source, such as animals, including humans, and plants. Exemplary samples including, but are not limited to: cervical epithelial cells (e.g., a sample obtained from a cervical swab); adenoid cells; anal epithelial cells; blood; blood products such as serum, plasma or buffy coat; saliva, cerebral spinal fluid, pleural fluid, milk, lymph, sputum and semen, and may be collected, for example, in Preservcyt, Surepath and/or Digene Collection Medium (“DCM”).

As used herein, the term “infectious agent” refers to any agent comprising a nucleic acid capable of infecting a plant or animal. By way of example and not limitation, the infectious agent may be a virus, including both DNA and RNA viruses; a viroid; a prokaryote, such as a bacterium and archaebacterium; a simple eukaryote, such as a fungus or a protozoa; or a multicellular organism, such as a mite and other parasite.

As used herein, “infectious agent nucleic acid” refers to any nucleic acid derived from the infectious agent. By way of example and not limitation, the infectious agent nucleic acid may be: comprised within the infectious agent; maintained in a host cell of the sample; integrated in the genome of a host cell of the sample; a product of expression of a gene of the infectious agent; or a product of replication of the infectious agent's genome. The infectious agent nucleic acid may be DNA or RNA.

As used herein, the term “hybridization assay” refers to an assay that separates the infectious agent nucleic acid from the sample by utilizing nucleic acids capable of hybridizing to the infectious agent nucleic acid. In one exemplary embodiment, the hybridization assay utilizes DNA:RNA hybrids to separate the infectious agent nucleic acid from the sample. In another exemplary embodiment, the DNA:RNA hybrids are formed utilizing nucleic acid capture probes bound to, or adapted to be bound to, a solid phase. In a further exemplary embodiment, the hybridization assay does not utilize amplification of the infectious agent nucleic acid. In a further exemplary embodiment, the hybridization assay does not utilize an exogenous nucleic acid polymerase to amplify a nucleic acid.

In a further exemplary embodiment, the hybridization assay comprises, consist, or consists essentially of a method of detecting a nucleic acid comprising: (1) providing a sample comprising a target nucleic acid; (2) providing at least one nucleic acid probe capable of hybridizing to the target nucleic acid, wherein said nucleic acid probe is DNA if the target nucleic acid is RNA and the nucleic acid probe is RNA if the target nucleic acid is DNA; (3) generating a DNA:RNA hybrid of the nucleic acid probe and the target nucleic acid; and (4) capturing the DNA:RNA hybrid to a solid phase. Exemplary solid phases include, but are not limited to: silica, borosilicates, silicates, anorganic glasses, organic polymers such as poly(meth)acrylates, polyurethanes, polystyrene, agarose, polysaccharides such as cellulose, metal oxides such as aluminum oxide, magnesium oxide, titanium oxide and zirconium oxide, metals such as gold or platinum, agarose, sephadex, sepharose, polyacrylamide, divinylbenzene polymers, styrene divinylbenzene polymers, dextrans, and derivatives thereof, and/or silica gels, beads, membranes, and resins; glass or silica surfaces, such as beads, plates, and capillary tubes; magnetizable or magnetic (e.g. paramagnetic, superparamagnetic, ferromagnetic or ferrimagnetic) particles, including but not limited to polystyrene, agarose, polyacrylamide, dextran, and/or silica materials having a magnetic material incorporated therein or associated therewith. In some exemplary embodiments, the nucleic acid probe can be linked to the surface of a processing vessel such as a micro-tube, a well of micro-plate, or capillary, and using these surfaces the infectious agent nucleic acid can be isolated on a micro scale. Where a biotinylated nucleic acid probe is provided, the solid phase may be coated with a substance capable of binding the biotin moiety, such as, for example, avidin, streptavidin, and/or neutravidin. In another embodiment, the solid phase may be coated with, or adapted to be coated with, an antibody specific for a DNA:RNA hybrid.

In one aspect, the infectious agent is a virus. In an exemplary embodiment, the virus is a human papillomavirus. In another exemplary embodiment, the infectious agent nucleic acid is a human papillomavirus DNA. Thus, in an aspect, a method for determining the viral load in a sample is provided, the method comprising: performing at least one hybridization assay with one or more human papillomavirus DNAs or fragments thereof in the sample; detecting resultant hybridized HPV DNA or fragments thereof; and calculating the viral load from detected hybridized HPV DNA or fragments thereof. In one exemplary embodiment, the hybridization assay of this method is the DIGENE® HC2 TEST™ (“HC2”). HC2 is an FDA approved screening test for 13 HPVs. This semi-quantitative assay is based on linear signal amplification with a limit-of-detection of approximately 5,000 copies of HPV DNA per assay. In the assay, unlabeled, full-length RNA probes are hybridized to denatured DNA targets in solution. The RNA:DNA hybrids are captured by specific antibodies conjugated to a 96-well microplate, then other hybrid-specific antibodies, labeled with alkaline phosphatase, are added to form a “sandwich”. The alkaline phosphatase-labeled antibodies allow semi-quantitative detection with a luminescent substrate. This assay is not affected by some components of clinical samples that can be inhibitory for target amplification assays. The HC2 assay, therefore, may be used to estimate the amount of HPV without extensive sample preparation. The dynamic range of the assay is approximately 3 to 4-logs. In another exemplary embodiment of this method, the sample is a cervical sample.

Infection of the host organism by the infectious agent can correlate with a diseased state of the host. Thus, in another aspect, a method for determining incidence of an infectious agent-related disease in a subject is provided, the method comprising: performing at least one hybridization assay with one or more infectious agent nucleic acid or fragment thereof derived from a sample from the subject; detecting resultant hybridized infectious agent nucleic acid or fragment thereof; calculating the load of the infectious agent in the sample from detected nucleic acid or fragment thereof; and correlating the load of the infectious agent in the sample to the incidence of the infectious agent-related disease.

In another aspect, a method for determining progression of a infectious agent-related disease in a subject is provided, the method comprising: providing a sample of diseased tissue from the subject at a first endpoint and a second endpoint; performing at least one hybridization assay with one or more infectious agent nucleic acid or fragment thereof in each sample of diseased tissue; detecting resultant hybridized infectious agent nucleic acid or fragment thereof; calculating the load of the infectious agent in the sample from each endpoint from the detected nucleic acid or fragment thereof; comparing the load of the infectious agent at the first endpoint to the load of the infectious agent at the second endpoint; and correlating the change in load of the infectious agent in the sample to the progression of the infectious agent-related disease.

By way of example and not limitation, cervical intraepithelial neoplasia (“CIN”) and cervical cancer correlate with infection by HPV, particularly hrHPV. Moreover, as disclosed herein, the cytological classification of CIN correlates with viral load of HPV. Thus, in another aspect, a method for determining incidence or progression of human papillomavirus-related disease in a subject is provided, the method comprising: performing at least one hybridization assay with one or more human papillomavirus DNAs or fragments thereof in the sample; detecting resultant hybridized human papillomavirus DNA or fragments thereof; calculating viral load from detected hybridized human papillomavirus DNA or fragments thereof; and correlating the viral load to the incidence or progression of the human papillomavirus-related disease.

In one exemplary embodiment, the hybridization assay comprises generating a DNA:RNA hybrid by a method comprising hybridizing a probe set comprising at least one RNA probe capable of hybridizing to at least one HPV DNA selected from the group consisting of HPV 16, HPV 18, HPV 31, HPV 33, HPV 35, HPV 39, HPV 45, HPV 51, HPV 52, HPV 56, HPV 58, HPV 59, and HPV 68 to the one or more human papillomavirus DNAs. In a further exemplary embodiment, said probe set comprises an RNA probe capable of hybridizing to an HPV 16 DNA, an RNA probe capable of hybridizing to an HPV 18 DNA, an RNA probe capable of hybridizing to an HPV 31 DNA, an RNA probe capable of hybridizing to an HPV 33 DNA, an RNA probe capable of hybridizing to an HPV 35 DNA, an RNA probe capable of hybridizing to an HPV 39 DNA, an RNA probe capable of hybridizing to an HPV 45 DNA, an RNA probe capable of hybridizing to an HPV 51 DNA, an RNA probe capable of hybridizing to an HPV 52 DNA, an RNA probe capable of hybridizing to an HPV 56 DNA, an RNA probe capable of hybridizing to an HPV 58 DNA, an RNA probe capable of hybridizing to an HPV 59 DNA, and an RNA probe capable of hybridizing to an HPV 68 DNA.

In one exemplary embodiment, a plasmid comprising an HPV genome or portion thereof may be used as a standard for estimating the HPV load in the sample. By way of example and not limitation, the standard may be a plasmid comprising the genome of HPV 16, HPV 18, HPV 31, HPV 33, HPV 35, HPV 39, HPV 45, HPV 51, HPV 52, HPV 56, HPV 58, HPV 59, of HPV 68, or a combination thereof. In a further exemplary embodiment, the standard comprises the genome of HPV 16 or HPV 18 or a portion thereof. In a further exemplary embodiment, the standard comprises, consists, or consists essentially of the genome of HPV 16 or a portion thereof.

Kits for performing the methods described herein are also contemplated.

In one aspect, the kit comprises at least one nucleic acid probe specific for the infectious agent nucleic acid. In another aspect, a solid phase may further be provided. By way of example and not limitation, the nucleic acid probe may be provided without further modification, may be adapted to be bound to a solid phase, such as by biotinylation, or may be provided bound to a solid phase. Any suitable solid phase may be used, including but not limited to: silica, borosilicates, silicates, anorganic glasses, organic polymers such as poly(meth)acrylates, polyurethanes, polystyrene, agarose, polysaccharides such as cellulose, metal oxides such as aluminum oxide, magnesium oxide, titanium oxide and zirconium oxide, metals such as gold or platinum, agarose, sephadex, sepharose, polyacrylamide, divinylbenzene polymers, styrene divinylbenzene polymers, dextrans, and derivatives thereof, and/or silica gels, beads, membranes, and resins; glass or silica surfaces, such as beads, plates, and capillary tubes; magnetizable or magnetic (e.g. paramagnetic, superparamagnetic, ferromagnetic or ferrimagnetic) particles, including but not limited to polystyrene, agarose, polyacrylamide, dextran, and/or silica materials having a magnetic material incorporated therein or associated therewith. In some exemplary embodiments, the nucleic acid probe can be linked to the surface of a processing vessel such as a micro-tube, a well of micro-plate, or capillary, and using these surfaces the infectious agent nucleic acid can be isolated on a micro scale. Where a biotinylated nucleic acid probe is provided, the solid phase may be coated with a substance capable of binding the biotin moiety, such as, for example, avidin, streptavidin, and/or neutravidin. In another embodiment, the solid phase may be coated with, or adapted to be coated with, an antibody specific for a DNA:RNA hybrid.

In another aspect, the kit may further comprise a standard comprising an infectious agent nucleic acid or portion thereof. Said standard may be provided as various dilutions, as a stock solution, or as a lyophilized standard or standards.

In another aspect, the kit may be used to determine the viral load in a sample, as a parameter by itself or as a indicator of the incidence or progression of an HPV-disease in a subject, said kit comprising: (1) a nucleic acid probe set comprising at least one nucleic acid probe capable of hybridizing to an HPV nucleic acid selected from the group consisting of HPV 16, HPV 18, HPV 31, HPV 33, HPV 35, HPV 39, HPV 45, HPV 51, HPV 52, HPV 56, HPV 58, HPV 59, of HPV 68, or a combination thereof; (2) optionally, a solid phase; and (3) optionally, a standard comprising a plasmid comprising the genome of HPV 16, HPV 18, HPV 31, HPV 33, HPV 35, HPV 39, HPV 45, HPV 51, HPV 52, HPV 56, HPV 58, HPV 59, of HPV 68, or a combination thereof. In a further exemplary embodiment, the standard comprises the genome of HPV 16 or a portion thereof.

### EXAMPLES

The information about the range and distribution of HPV load in clinical specimens is important for the development of accurate clinical tests. Determination of the load of HPV in a sample may be a predictor of the incidence or progression of HPV-related diseases.

The amount of HPV in cervical specimens was estimated using HC2. This test is a semi-quantitative assay based on linear signal amplification with a limit-of-detection of approximately 5000 HPV copies per assay and 3 to 4-log dynamic range. The dynamic range of the assay was extended by a serial dilution strategy.

Two large sets of HPV positive specimens (n=501 and 569) were analyzed and 9% to 11% of specimens were shown to contain more than more than 5×10⁷ copies of HPV. The HPV load was also assessed for the set of specimens with known cytology diagnoses (n=9435). The percentage of specimens with more than 5×10⁷ copies of HPV was 0.89 for WNL, 4.2 for ASCUS, 14.31 for LSIL and 22.24 for HSIL specimens. Information about the distribution of HPV load within each group is useful for HPV assay design and automation, where cross-reactivity and sample-to-sample contamination are concerns.

The HPV load in a large number of archived, HPV positive, cervical specimens was determined using the HC2 assay. The dynamic range (upper limit) of the assay was extended by a serial dilution strategy. The study of HPV load was conducted on residual specimens, or specimen data, selected from an archival collection that included HPV positive clinical specimens from normal populations in California, China, Maryland, and Texas. The California (n=501) specimens were collected in QIAGEN Sample Transport Medium (“STM”). The specimens from China (n=569) were collected in PRESERVCYT medium (Hologic, Inc., Bedford, Mass.) (“PC”). Specimens from Maryland and Texas (n=9435, combined), for which the cytology was known, were collected in PC. Other samples consisted of serial dilutions of HPV 16 plasmids in STM.

The HPV DNA was assessed using HC2 according to the manufacturer's instructions. The sample aliquot volume of specimen input to the assay was 50 μL for the STM specimens and 2 mL for the PC specimens. The RLU readings from the various cytology groups were compared using the Wilcoxon/Kruskal-Wallis Rank Sum Test with chi-square approximation (JMP8, SAS).

### Example 1

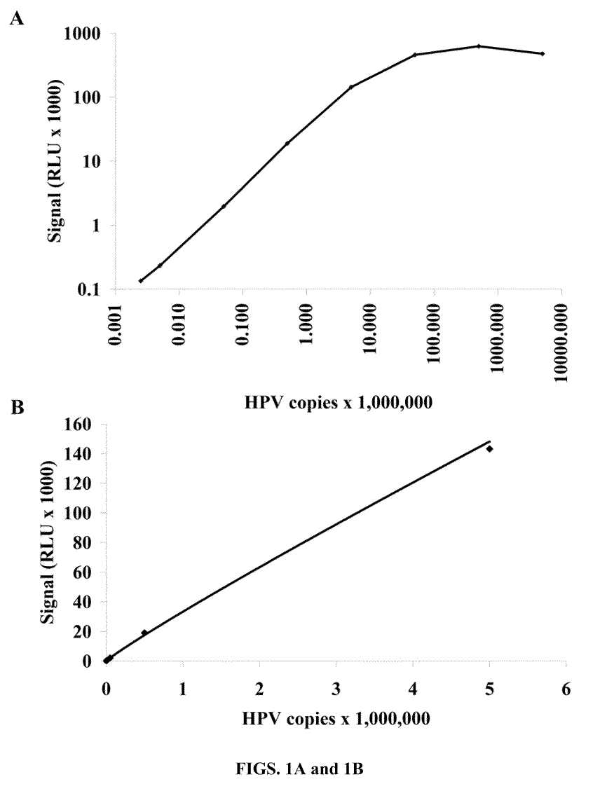

The abundance of HPV in serial dilutions of HPV 16 plasmid was determined using the HC2 TEST. The dependence of signal in relative luminescent units (RLU) on HPV plasmid concentration, copies per assay, is presented in FIG. 1A. The signal increased with copy number in a linear relationship across approximately 3.5 logs of HPV concentration, from 2,500 to 5×10⁶ copies per assay. The slope of the line begins to curve downward at 5×10⁶ copies, with a plateau thereafter. The signal intensity that corresponds to this point of inflection was between 300,000 RLU to 400,000 RLU. The peak signal was approximately 600,000 RLU for 5×10⁸ copies.

Good linearity (R²=0.999) was obtained for the standard curve with a trend line described by y=0.0287x+55, where x=copy number and y=RLU (FIG. 1B); data points include x values of 2500, 5000, 50000, 500000, 5000000 and y values of 134, 234, 1962, 19008, 143114, respectively. A similar standard curve was obtained for HPV 18 plasmids. Thus, the dynamic range is expected to be similar for all HPV types. Based on this standard curve, samples with signals above 300,000 RLU to 400,000 RLU are no longer within linear range and are diluted for copy number calculation by the HC2 assay.

In sum, HC2 may be used to generate a linear standard curve of HPV copy number covering 3.5 logs of HPV concentration, thus indicating that HC2 is useful for estimating HPV copy number in a sample.

### Example 2

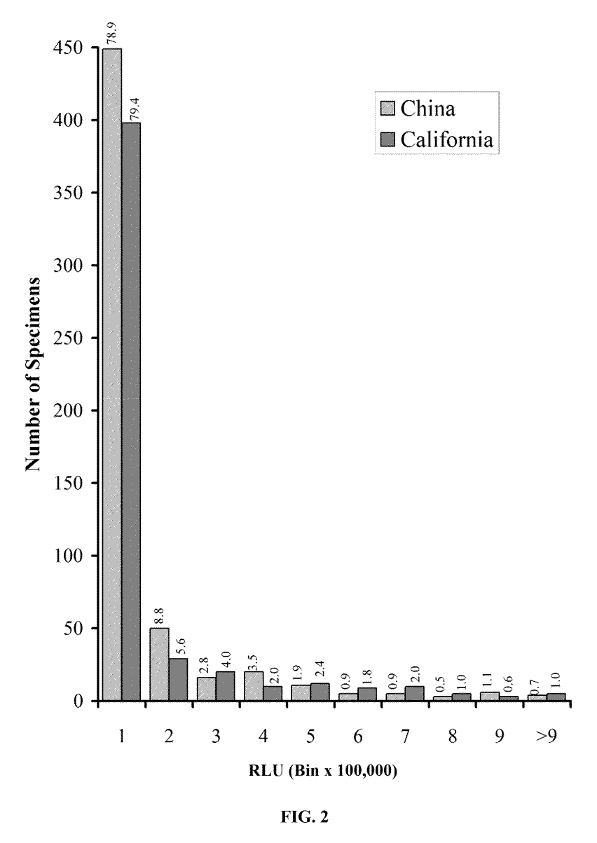

Two sets of cervical samples from normal populations from California (n=501) and China (n=569) were analyzed by HC2 to estimate how many specimens exceeded the upper limit of the linear range of the assay, which was between 300,000 RLU to 400,000 RLU. This corresponds to the approximate number of specimens containing more than 5×10⁶ copies of HPV per assay. The signal intensities (RLU) resulting from HC2 assays for two sets of samples were sorted into distribution histograms with bins of 100,000 RLU. The California population included 11% of specimens above 300,000 RLU and 9% above 400,000 RLU (FIG. 2). For the China population, 9% of specimens were above 300,000 RLU and 6% of specimens were above 400,000 RLU. Based on the data, approximately 9% to 11% of HPV positive specimens in a normal population will include more than 5×10⁶ copies of HPV per assay. This equates to 5×10⁷ copies per specimen if 1/10^{th} of the specimen is used per assay, as it is for a 20-mL PC specimen.

Because the clinical specimens resulting in RLU greater than 400,000 do not fall within the linear range of the assay, the copy number may be estimated from a standard curve after the sample is diluted to within the linear range of the assay. To demonstrate this, the signal (RLU) was measured for serial dilutions of six STM specimens with a high RLU values. The serial dilutions included 1:25, 1:50, 1:100, 1:200, 1:400, 1:800 and 1:1600. Three replicates of each dilution were tested. The replicate sample input volume was 50 μL. The original, total volume of the specimen was 1 mL (STM). A serial dilution of HPV 16 plasmid was run on the same assay plate as the specimen dilutions.

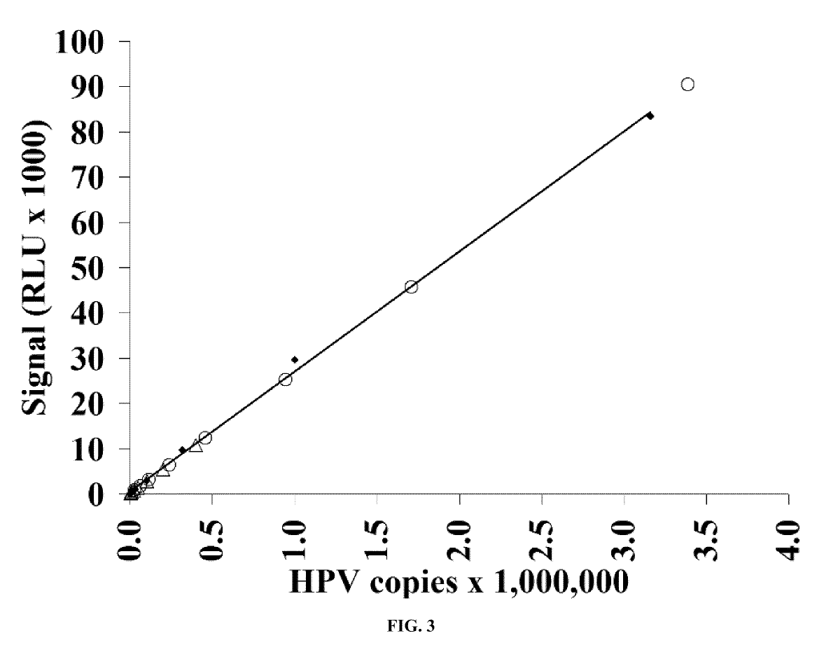

The results for two of the clinical samples are presented in FIG. 3. A line was fitted to the plasmid standard curve (y=0.026x+66). The HPV copy number per serial dilution was calculated from the standard curve. For each of the six specimens, the HPV copy number per assay (assuming 50 μL of sample input) of the undiluted specimen was estimated by multiplying the copy number for each dilution by its dilution factor. From this, the total number of HPV copies per specimen (1 mL STM) was calculated and shown to fall between 7×10⁷ and 2×10⁹ copies (Table 1).

TABLE 1
Estimates of HPV copy number per specimen
for six specimens with a high HC2 signal.

| | | | Calculated HPV | Calculated HPV |
|---|---|---|---|---|
| Specimen | | | copy number | copy number |
| ID | RLU | RLU/CO | per assay | per specimen |
| 360 | 951652 | 1889 | 1.85 × 10⁸ | 3.7 × 10⁹ |
| 664 | 581436 | 1501 | 1.99 × 10⁷ | 3.98 × 10⁸ |
| 1079 | 399672 | 1085 | 1.83 × 10⁷ | 3.66 × 10⁸ |
| 495 | 784436 | 1379 | 9.93 × 10⁶ | 1.98 × 10⁸ |
| 1403 | 486404 | 995 | 7.37 × 10⁶ | 1.47 × 10⁸ |
| 890 | 513139 | 1216 | 6.64 × 10⁶ | 1.33 × 10⁸ |

Values of RLU and RLU/CO (CO, cut-off is RLU for 1 pg/ml of HPV 16 plasmid) are for undiluted specimens and HPV copy number is calculated from the serial dilution of specimens.

In sum, approximately 90% of clinical samples fall within the linear range of the HC2 assay. Moreover, the range of detection in the sample may be extended by serial dilution of the sample. Thus, HC2 is useful for calculating the viral load in clinical samples.

### Example 3

The HPV load was also assessed for another set of samples (n=9435) for which cytology diagnoses were known. Normal cytological evaluations are deemed WNL (within normal limits), while abnormal cytological evaluations are separated into three categories of cervical epithelial neoplasia: ASCUS (atypical squamous cells of unknown significance), LSIL (low grade squamous intraepithielial lesion) and HSIL (high grade squamous intraepithielial lesion).

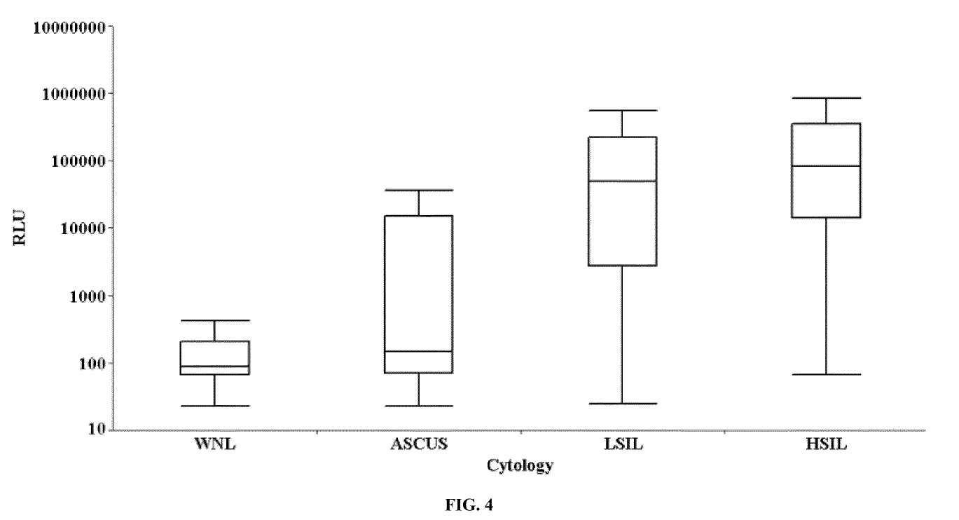

The present assessment was based on the RLU signal resulting from the HC2 TEST. The percentage of specimens above 400,000 RLU was 5.12 for all diagnoses, 0.89 for WNL (within normal limits), 4.2 for ASCUS (atypical squamous cells of unknown significance), 14.31 for LSIL (low grade squamous intraepithielial lesion) and 22.24 for HSIL (high grade squamous intraepithielial lesion). The median RLU for the different groups was: 90 for WNL (viral load ˜1.2×10³), 149.5 for ASCUS (viral load ˜3.3×10³), 49225.5 for LSIL (viral load ˜1.7×10⁶) and 83630 for HSIL (viral load ˜2.9×10⁶) (approximate viral loads calculated according to the trend line of FIG. 1B, y=0.0287x+55). See FIG. 4.

Statistical differences in RLU among the groups were determined using the Wilcoxon/Kruskal-Wallis Rank Sum test. There was a significant difference in RLU among the diagnoses (p<0.0001), despite a large variation in RLU within each diagnosis group. Furthermore, there was a significant increase in the number of specimens above 300,000 RLU (p=0.0005) and above 400,000 RLU (p=0.04) for increasing severity of lesion. The lower p-value for the specimens above 400,000 RLU, as compared to those above 300,000 RLU, indicates a reduced difference in RLU among the cytology groups. Without being bound by theory, this may reflect the lack of discrimination of the assay at the upper limit. These data indicate that the percent of specimens above a given signal threshold increase with the prevalence of advanced disease (for example, HSIL) in a population.

The dynamic range of the HC2 assay was demonstrated to be 2500 copies to 5×10⁶ copies of HPV plasmid per assay, which agrees with other published results. Some specimens of any study may contain a HPV load that is higher than the upper limit of the HC2 dynamic range. The HPV copy number for these out-of-range specimens is not a function of the resulting RLU but may be estimated by comparing serial dilutions of the specimens with that of known concentrations of HPV plasmids. Thus, the use of HC2 to calculate HPV load is feasible, even when the HPV load is high. Determination of HPV load by HC2 may be especially useful when it is not convenient to use the more common quantitative PCR technique or when substances inhibitory to PCR are present in clinical samples.

Two sets of clinical samples obtained from general screening populations both indicated that approximately 9% to 11% of specimens have a HPV load above 400,000 RLU, or 5×10⁶ copies per assay. This estimate of HPV copy number was calculated from the equation of the standard curve for the HPV 16 plasmid. Although many studies report HPV viral load, only a few provide or allow the calculation of the amount of HPV per specimen.

For example, one study reported the distribution of HPV load and the distribution of cells per assay for 40 specimens, but paired values for each specimen were not given. Using the data from that study, the percent of specimens above 5×10⁶ copies per assay was calculated to be 9.9%, which is similar to other results shown in this report. The percent of specimens above 1×10⁷ copies per assay was figured to be 3.7%. Furthermore, a hypothetical, maximum load within a population may reach 1×10¹¹ copies per specimen if the number of copies per cell is 1×10⁴ and the number of cells per specimen is 1×10⁷.

The percent of specimens containing 5×10⁷ copies of HPV DNA or more may increase for populations with a higher prevalence of HPV or severe lesions. This may occur, for example, in colposcopy-referral populations or in populations with a high prevalence of HIV infection. The analysis of the archive data showed that the median RLU increases for increasing severity of disease in WNL, ASCUS, LCIL and HSIL groups. Despite this correlation between RLU and cytological diagnoses, the distribution of viral load within each group remains very broad. Other published data report this direct relationship, but some studies report no increase or a decrease in load with severity of disease. This broad distribution of viral load within each group complicates the use of this parameter as a biomarker.

It is relevant to consider the potential frequency of a high load when designing a specific and accurate clinical assay for HPV. The HPV in specimens may include multiple, highly homologous types. For example, the overall identity between HPV 16 and HPV 31 is 71%, and that between HPV 18 and HPV 45 is 81% (Clustal-X alignment). The analytical specificity of detection probes for one HPV type depends, in part, on the concentration of the potential cross-reactive types.

Another instance where the knowledge of the viral load may be important is in assay automation of liquid handling. Cross-contamination between clinical samples needs to be addressed with an understanding of the HPV distribution within a set of clinical samples. High levels of HPV load present a challenge to assay design and automation, for which cross-reactivity and sample-to-sample contamination are significant concerns.

In sum, the severity of a cervical lesion may be estimated by calculating the viral load using HC2. As can be seen at FIG. 3 and FIG. 4, a viral load of approximately 1×10⁴ is indicative of CIN classified as ASCUS, LSIL, or HSIL, while a viral load of 1×10⁶ or greater is indicative of CIN classified as LSIL and HSIL. Although there is some overlap, a viral load of from 1×10⁴ to 1×10⁵ correlates more closely with ASCUS than with LSIL and HSIL. These correlations indicate that calculation of viral loads according to the present methods is useful for triaging patients who may require further monitoring, particularly in regions where cytological diagnostic resources may be scarce.

What is claimed is:

1. A method for determining a load of an infectious agent in a sample, the method comprising:
performing at least one hybridization assay with a nucleic acid of the virus or fragment thereof in the sample to form a hybridized infectious agent nucleic acid;
detecting the hybridized infectious agent nucleic acid; and
calculating the load of the infectious agent from the detected hybridized infectious agent nucleic acid.

2. The method of claim 1 wherein said hybridization assay comprises forming a DNA:RNA hybrid.

3. The method of claim 1 wherein said infectious agent is a virus and said infectious agent nucleic acid is a viral nucleic acid.

4. The method of claim 3 wherein said viral nucleic acid is a DNA or an RNA.

5. The method of claim 3 wherein said virus is a high-risk human papillomavirus.

6. The method of claim 4 wherein said viral nucleic acid is a high-risk human papillomavirus DNA.

7. The method of claim 1 wherein said sample is a cervical sample.

8. A method for determining the viral load in a sample, the method comprising
performing at least one hybridization assay with one or more human papillomavirus DNAs or fragments thereof in the sample;
detecting resultant hybridized HPV DNA or fragments thereof; and
calculating the viral load from detected hybridized HPV DNA or fragments thereof.

9. The method of claim 8 wherein detecting resultant hybridized HPV DNA or fragments thereof comprises generating a detectable signal from the hybridized HPV DNA and measuring the intensity of the detectable signal.

10. The method of claim 9 wherein calculating the viral load comprises:
a. generating a standard curve with more than one known concentration of an HPV nucleic acid; and
b. calculating the viral load of the sample based on the standard curve.

11. The method of claim 10, wherein calculating the viral load of the sample is based on a linear portion of the standard curve.

12. A method of comparing the incidence of a human papillomavirus-related disease state among more than one population of subjects, said method comprising
determining the viral load of a representative number of subjects of each population according to the method of claim 8;
calculating the percentage of subjects in each population having a viral load above a threshold; and
correlating the percentage of subjects in each population to the relative incidence of the human papillomavirus-related disease state.

13. The method of claim 12, wherein said of the human papillomavirus-related disease is cervical intraepithelial neoplasia (CIN).

14. The method of claim 13, wherein the percentage of subjects of the population having a viral load of 1×10⁴ or greater correlates to the relative incidence of CIN classified as atypical squamous cells of unknown significance (ASCUS), low grade squamous intraepithielial lesion (LSIL), and high grade squamous intraepithielial lesion (HSIL).

15. The method of claim 13, wherein the percentage of subjects of the population having a viral load of 1×10⁶ or greater correlates to the relative incidence of CIN classified as atypical squamous cells of unknown significance (ASCUS), low grade squamous intraepithielial lesion (LSIL), and high grade squamous intraepithielial lesion (HSIL).

16. A method of triaging more than one cervical sample for cytological evaluation, said method comprising:
determining the viral load of each cervical sample according to the method of claim 8; and
triaging the sample for cytological evaluation according to viral load, wherein samples with the highest viral load are selected first for cytological evaluation.

17. A method for predicting incidence or progression of human papillomavirus-related disease state in a subject, the method comprising
performing at least one hybridization assay with one or more human papillomavirus DNAs or fragments thereof in the sample;
detecting resultant hybridized human papillomavirus DNA or fragments thereof;
calculating viral load from detected hybridized human papillomavirus DNA or fragments thereof; and
correlating the viral load to the incidence or progression of the human papillomavirus-related disease state.

18. The method of claim 17, wherein said human papillomavirus-related disease state is cervical intraepithelial neoplasia.

19. The method of claim 18 wherein a viral load of approximately 1×10⁴ or greater is predictive of cervical intraepithelial neoplasia (CIN) classified as atypical squamous cells of unknown significance (ASCUS), low grade squamous intraepithielial lesion (LSIL), and high grade squamous intraepithielial lesion (HSIL).

20. The method of claim 18, wherein a viral load of approximately 1×10⁴ to approximately 1×10⁵ is predictive of a CIN classified as ASCUS.

21. The method of claim 18, wherein a viral load of approximately 1×10⁶ or greater is predictive of a CIN classified as LSIL or HSIL.
